(19) United States
(12) Patent Application Publication
(10) Pub. No.: US 2026/0108765 A1
(43) Pub. Date: Apr. 23, 2026

(54) INCLINED PROJECTION BASED STRUCTURE OF SURGICAL INSTRUMENT FOR IRRADIATING FOCUSED ULTRASOUND WAVE AT PLURALITY OF DEPTHS AND SURGICAL METHOD USING THE SAME

(71) Applicant: Shenb Co., Ltd., Seoul (KR)

(72) Inventor: Sunyoung KANG, Seoul (KR)

(21) Appl. No.: 18/955,637

(22) Filed: Nov. 21, 2024

(30) Foreign Application Priority Data
Oct. 18, 2024 (KR) 10-2024-0142929

(51) Int. Cl. A61N 7/00

(52) U.S. Cl. CPC A61N 7/00

(57) ABSTRACT

According to an exemplary embodiment, a structure of a surgical instrument for irradiating a focused ultrasound wave at a plurality of depths includes a movement unit, one pair of frames, a first transducer assembly which horizontally moves by the movement unit and upwardly/downwardly vertically moves to irradiate a focused ultrasound wave at a plurality of depths, a fitting unit which is coupled with the first transducer assembly, and a housing which accommodates the first transducer assembly in an inner space and is coupled to the movement unit to horizontally move the first transducer assembly in one direction or in the other direction by the movement unit or horizontally reciprocates the first transducer assembly in one direction and the other direction.

### CROSS-REFERENCE TO RELATED APPLICATIONS

This application claims the priority of Korean Patent Application No. 10-2024-0142929 filed on Oct. 18, 2024, in the Korean Intellectual Property Office, the disclosure of which is incorporated herein by reference.

### BACKGROUND

### Field

The present disclosure relates to an inclined projection based structure of surgical instrument for irradiating a focused ultrasound wave at a plurality of depths and a surgical method using the same, and more particularly, to an inclined projection based structure of surgical instrument which simply irradiates a focused ultrasound wave to a skin of a subject at a plurality of depths based on upward/downward vertical movement of a transducer assembly when sliding according to a pressure after contact between an inclined projection and a fitting groove while horizontal movement and a surgical method using the same.

### Description of the Related Art

In the related art, there is a motor structure for linear movement of a transducer. However, in order to implement upward/downward movement of the transducer, an actuator, such as a separate solenoid or motor to adjust a depth of the transducer, a sensor for measuring upward/downward movement distance of the transducer in the case of a piezo or ball screw type, needs to be additionally configured. Therefore, the configuration of the device with the transducer becomes complex.

In the related art, there is a surgical method using a surgical instrument which irradiates a focused ultrasound wave to a skin of a subject at a plurality of depths. However, according to the surgical method of the related art, when the focused ultrasound wave is irradiated onto the skin of the subject at a plurality of depths, first, a transducer assembly moves up/down at a first depth, and then the transducer horizontally linearly irradiates the focused ultrasound wave, and then, the transducer assembly is adjusted up/down, and then horizontally and linearly irradiates the focused ultrasound wave at a second depth to improve a skin state of the subject. A plurality of motors is required to move up/down the transducer assembly and horizontally and linearly radiate the focused ultrasound wave. Therefore, there are problems in that a configuration of a surgical instrument and a process of a surgical method become complex.

Accordingly, studies for a surgical instrument and a surgical method for simply irradiating an ultrasound wave to a skin of a subject at a plurality of depths are required

### RELATED ART DOCUMENT

### Patent Document

Korean Registered Patent Publication No. 10-1712024 (Registered on Feb. 24, 2017).

### SUMMARY

Accordingly, the present disclosure has been devised to solve the above-described problems and an object to be achieved by the present disclosure is to provide an inclined projection based structure of a surgical instrument for irradiating a focused ultrasound wave at a plurality of depths which adjusts an irradiation depth of a focused ultrasound wave by implementing an upward/downward vertical movement of a transducer assembly while omitting a configuration of an actuator for horizontal movement and upward/downward vertical movement and sets an irradiation depth of the focused ultrasound wave without adding a separate sensor.

Further, another object of the present disclosure is to provide an inclined projection based structure of a surgical instrument which accepts the surgical method of the related art and irradiates a focused ultrasound wave onto a skin of a subject at a plurality of depths based on the upward/downward vertical movement which occurs when an inclined projection and a fitting groove are in contact with each other and then slide according to a pressure while the transducer assembly horizontally moves and a surgical method using the same.

However, technical objects to be achieved in the present disclosure are not limited to the aforementioned technical objects, and another not-mentioned technical object will be clearly understood by those skilled in the art from the description below.

In order to achieve the above-described objects, according to an aspect of the present disclosure, an inclined projection based structure of a surgical instrument for irradiating a focused ultrasound wave at a plurality of depths may include a movement unit; one pair of frames including a plurality of inclined projections to be opposite to each other, through which the movement unit passes; a first transducer assembly which horizontally moves by the movement unit and upwardly/downwardly vertically moves to irradiate a focused ultrasound wave at a plurality of depths; a fitting unit which is coupled with the first transducer assembly and includes a plurality of fitting grooves which upwardly or downwardly slides by a pressure after being contact with the inclined projection according to a horizontal movement degree of the first transducer assembly and then is fitted with the inclined projection to implement the upward/downward vertical movement of the first transducer assembly; and a housing which accommodates the first transducer assembly in an inner space and is coupled to the movement unit to horizontally move the first transducer assembly in one direction or in the other direction by the movement unit or horizontally reciprocates the first transducer assembly in one direction and the other direction and encloses a part of the first transducer assembly accommodated in an inner space with a spring which expands or contracts by upward or downward sliding of the fitting unit to implement upward/downward vertical movement of the first transducer assembly.

Further, the pair of frames may include: a first frame including a first inclined projection with an upwardly inclined surface to upwardly vertically move the first transducer assembly; and a second frame including a second inclined projection having a downwardly inclined surface provided in an inside opposite to the first frame to downwardly vertically move the second transducer assembly.

Further, in order to upwardly vertically move the first transducer assembly, when the first transducer assembly horizontally moves in one direction by the movement unit in a state in which the first transducer assembly downwardly vertically moves to a lowermost point, if the first inclined projection is in contact with a first fitting groove formed at one side, the fitting unit may upwardly slide according to the pressure.

Further, when the first inclined projection is fitted into the first fitting groove, in the housing, the spring may expand to allow the first transducer assembly upwardly vertically moves to the uppermost point.

Further, in order to downwardly vertically move the first transducer assembly, when the first transducer assembly horizontally moves in the other direction by the movement unit in a state in which the first transducer assembly upwardly vertically moves to an uppermost point, if the second inclined projection is in contact with a second fitting groove formed at the other side, the fitting unit downwardly slides according to the pressure.

Further, when the second inclined projection is fitted into the second fitting groove, in the housing, the spring contracts to allow the first transducer assembly to downwardly vertically move to the lowermost point.

Further, the first transducer assembly may include an elastic projection at a part of the outside to maintain a state which upwardly vertically moves to the uppermost point or downwardly vertically moves to the lowermost point.

Further, the housing may include a first locking groove through which a projection of the first transducer assembly passes to maintain the first transducer assembly in an upwardly vertically moved state to the uppermost point when the first transducer assembly upwardly vertically moves to the uppermost point; and a second locking groove through which a projection of the first transducer assembly passes to maintain the first transducer assembly in a downwardly vertically moved state to the lowermost point when the first transducer assembly downwardly vertically moves to the lowermost point.

Further, the movement unit rotates with the power received from a motor to horizontally move the first transducer assembly, and the structure further includes a horizontal guide which is coupled to the housing to guide a horizontal movement of the first transducer assembly to allow the first transducer assembly to horizontally move and block the rotation by the movement unit based on a time when the movement unit rotates with the power transmitted from the motor.

Further, the surgical instrument may be a cartridge and a handpiece to which the structure is applicable.

Further, when the structure is applied to the cartridge, the structure serves as a traction shaft which allows the first transducer assembly to move a transducer which irradiates a focused ultrasound wave using a driving force of a motor embedded in the handpiece and the transducer is embedded in the cartridge and is coupled with the first transducer assembly and horizontally moves and upwardly/downwardly vertically moves by following the horizontal movement and upward/downward vertical movement of the first transducer assembly to irradiate the focused ultrasound wave at a plurality of depths.

Further, when the structure is applied to the handpiece, the structure serves as a first traction shaft which allows the first transducer assembly to move a transducer which irradiates a focused ultrasound wave using a driving force of a motor embedded in the handpiece and is coupled with a second transducer assembly included in the cartridge, and the second transducer assembly serves as a second traction shaft which moves the transducer by following the horizontal movement and upward/downward vertical movement of the first transducer assembly, and the transducer is embedded in the cartridge and is coupled with the second transducer assembly and horizontally moves and upwardly/downwardly vertically moves by following the horizontal movement and upward/downward vertical movement of the second transducer assembly to irradiate the focused ultrasound wave at a plurality of depths.

In the cartridge, a transmission opening which allows a focused ultrasound wave to pass through a contact surface which is in contact with the skin of the subject is formed to irradiate a focused ultrasound wave generated from the transducer onto the skin of the subject.

In order to allow the first transducer assembly to move and be coupled with the second transducer assembly, in the handpiece, a binding opening with a diameter through which the first transducer assembly or the second transducer assembly passes to be movable is formed on a binding surface with the cartridge.

Further, according to another aspect of the present disclosure, a surgical method using an inclined projection based structure of a surgical instrument for irradiating a focused ultrasound wave at a plurality of depths includes a) a step of horizontally moving a first transducer assembly accommodated in an inner space of a housing in one direction by a movement unit coupled to the housing in a state in which the first transducer assembly is downwardly vertically moved to the lowermost point; b) a step of horizontally reciprocating the first transducer assembly if an inclined projection of a frame through which the movement unit passes is not in contact with a fitting groove of a fitting unit coupled with the first transducer assembly based on a time when the horizontal movement of the first transducer assembly in one direction is completed; c) a step of upwardly vertically moving the first transducer assembly by the fitting unit which upwardly slides by the expansion of a spring embedded in the housing by the pressure after the inclined projection is in contact with the fitting groove when the inclined projection is in contact with the fitting groove based on a time when the horizontal movement of the first transducer assembly in one direction is completed; d) a step of upwardly vertically moving the first transducer assembly to the uppermost point as the fitting unit upwardly slides, after the step c); e) a step of horizontally moving the first transducer assembly which completes the upward vertical movement, in the other direction, by the movement unit; f) a step of horizontally reciprocating the first transducer assembly if the inclined projection is not in contact with the fitting groove based on a time when the horizontal movement of the first transducer assembly in the other direction is completed; g) a step of downwardly vertically moving the first transducer assembly by the fitting unit which downwardly slides by the contraction of the spring by the pressure after the inclined projection is in contact with the fitting groove when the inclined projection is in contact with the fitting groove based on a time when the horizontal movement of the first transducer assembly in the other direction is completed; h) a step of upwardly vertically moving the first transducer assembly to the lowermost point as the fitting unit downwardly slides, after the step g); and i) a step of horizontally moving and upwardly/downwardly vertically moving the first transducer assembly to irradiate the focused ultrasound wave at a plurality of depths by repeating the steps a) to h) until a skin treatment of a subject is completed.

According to the present disclosure, a surgical instrument with a simple configuration including a structure which adjusts an irradiation depth of a focused ultrasound wave by implementing an upward/downward vertical movement of a transducer assembly while omitting a configuration of an actuator for horizontal movement and upward/downward vertical movement and sets an irradiation depth of the focused ultrasound wave without adding a separate sensor is provided.

Further, the present disclosure accepts the surgical method of the related art and a focused ultrasound wave is irradiated onto a skin of a subject at a plurality of depths based on the upward/downward vertical movement which occurs when an inclined projection and a fitting groove are in contact with each other and then slide according to a pressure while the transducer assembly horizontally moves to improve a skin state of the subject based on various surgical methods.

However, a technical object to be achieved in the present disclosure is not limited to the aforementioned effects, and another not-mentioned effects will be obviously understood by those skilled in the art from the description below.

### BRIEF DESCRIPTION OF THE DRAWINGS

The above and other aspects, features and other advantages of the present disclosure will be more clearly understood from the following detailed description taken in conjunction with the accompanying drawings, in which:

FIG. 1 is a block diagram of components which configure an inclined projection based structure of a surgical instrument for irradiating a focused ultrasound wave at a plurality of depths according to an exemplary embodiment of the present disclosure;

FIG. 2 is a schematic diagram of an inclined projection based structure of a surgical instrument for irradiating a focused ultrasound wave at a plurality of depths according to an exemplary embodiment of the present disclosure;

FIG. 3 is a view illustrating a fitting process of a first inclined projection and a first fitting groove for upward vertical movement of a first transducer assembly;

FIG. 4 is a view illustrating a fitting process of a second inclined projection and a second fitting groove for downward vertical movement of a first transducer assembly;

FIG. 5 is a view for explaining horizontal movement and upward/downward vertical movement of a first transducer assembly;

FIG. 6 is a schematic diagram of an inclined projection based structure of a surgical instrument for irradiating a focused ultrasound wave at a plurality of depths according to another exemplary embodiment of the present disclosure; and

FIG. 7 is a flowchart illustrating processes of a surgical method using an inclined projection based structure of a surgical instrument for irradiating a focused ultrasound wave at a plurality of depths according to an exemplary embodiment of the present disclosure.

### DETAILED DESCRIPTION OF THE PREFERRED EMBODIMENT

Hereinafter, exemplary embodiments of the present disclosure will be described more fully with reference to the accompanying drawings for those skilled in the art to easily implement the present disclosure. Description of the present disclosure is just an embodiment for structural and functional description so that the scope of the present disclosure is not interpreted to be limited by the embodiment described in the specification. That is, the embodiment may be modified in various forms so that it is understood that the scope of the present disclosure has equivalents which are capable of implementing the technical spirit. Further, it does not mean that the specific embodiment includes the object or effect proposed in the present disclosure or includes only the effect so that it is not understood that the scope of the present disclosure is limited thereby.

In the meantime, meanings of terms described in the present disclosure may be understood as follows.

The terms “first” or “second” are used to distinguish one component from the other component so that the scope should not be limited by these terms. For example, a first component may be referred to as a second component, and similarly, a second component may be referred to as a first component. It should be understood that, when it is described that an element is “connected” to another element, the element may be directly connected to the other element or connected to the other element through a third element. In contrast, it should be understood that, when it is described that an element is “directly connected” to another element, no element is present between the element and the other element. In the meantime, other expressions which describe the relationship between components, that is, “between” and “directly between”, or “adjacent to”and “directly adjacent to”need to be interpreted by the same manner.

Unless the context apparently indicates otherwise, singular expressions should be understood to include plural expressions, and it should be understood that terms “include” or “have” indicate that a feature, a number, a step, an operation, a component, a part or the combination thereof described in the specification is present, but do not exclude a possibility of presence or addition of one or more other features, numbers, steps, operations, components, parts or combinations thereof, in advance.

Unless they are contrarily defined, all terms used herein including technological or scientific terms have the same meaning as those generally understood by a person with ordinary skill in the art. Terms which are defined in a generally used dictionary should be interpreted to have the same meaning as the meaning in the context of the related art but are not interpreted as an ideally or excessively formal meaning if it is not clearly defined in the present disclosure.

### STRUCTURE OF SURGICAL INSTRUMENT

### 1) First Embodiment

Hereinafter, a first embodiment of an inclined projection based structure 100 of a surgical instrument for irradiating a focused ultrasound wave at a plurality of depths which is the present disclosure will be described in detail with reference to accompanying drawings.

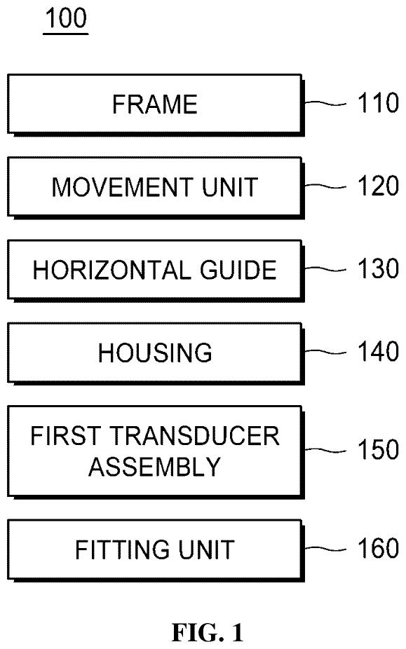

FIG. 1 is a block diagram of components which configure an inclined projection based structure of a surgical instrument for irradiating a focused ultrasound wave at a plurality of depths according to an exemplary embodiment of the present disclosure.

Referring to FIG. 1, according to the first embodiment, the structure 100 of the present disclosure includes a frame 110, a movement unit 120, a horizontal guide 130, a housing 140, a first transducer assembly 150, and a fitting unit 160 to allow a surgical instrument to irradiate an ultrasonic wave to a skin of a subject at a plurality of depths.

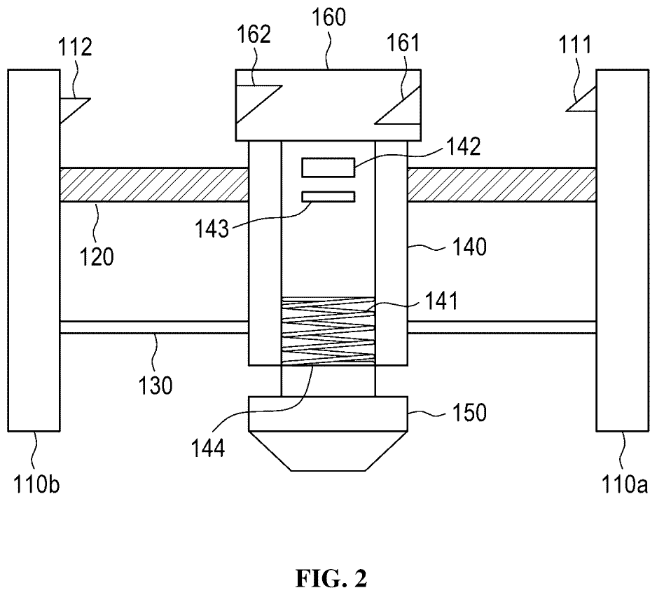

The components 110 to 160 of the structure 100 may be embedded in the surgical instrument and may be implemented in the form of the example illustrated in FIG. 2 to be embedded in the surgical instrument.

FIG. 2 is a schematic diagram of an inclined projection based structure of a surgical instrument for irradiating a focused ultrasound wave at a plurality of depths according to an exemplary embodiment of the present disclosure.

Referring to FIG. 2, the frame 110 of the present disclosure is configured by a first frame 110a and a second frame 110b which are one pair of frames through which the movement unit 120 passes to be coupled with both ends of the horizontal guide 130.

At this time, the interval between the inside of the first frame 110a and the inside of the second frame 110b is equal to the maximum horizontal movement distance of the first transducer assembly 150.

That is, the horizontal movement distance of the first transducer assembly 150 means a linear movement distance from the inside of the first frame 110a to the inside of the second frame 110b or a linear movement distance from the inside of the second frame 110b to the inside of the first frame 110a.

A first inclined projection 111 is formed on the inside of the first frame 110a which is opposite to the second frame 110b.

The first inclined projection 111 is provided in the first frame 110a to have an upwardly inclined surface 111a to allow upward vertical movement of the first transducer assembly 150.

A second inclined projection 112 is formed on the inside of the second frame 110b which is opposite to the first frame 110a.

The second inclined projection 112 is provided in the second frame 110b to have a downwardly inclined surface 112a to allow downward vertical movement of the first transducer assembly 150.

The movement unit 120 of the present disclosure is coupled to the first and second frames 110a and 110b to horizontally move the housing 140 and the first transducer assembly 150 accommodated in the housing 140.

At this time, as long as the movement unit 120 horizontally moves the housing 140 and the first transducer assembly 150, a movement method is not limited. However, according to the exemplary embodiment, the movement unit may be a ball screw which is coupled with a motor (not illustrated) and rotates with a power transmitted from the motor to horizontally move the housing 140 and the first transducer assembly 150.

The horizontal guide 130 of the present disclosure refers to a bar which is coupled with the housing 140 to guide horizontal movement of the housing 140 and the first transducer assembly 150 when the movement unit 120 is implemented as a ball screw as described above.

The horizontal guide 130 may be coupled to the housing 140 to prevent the housing 140 and the first transducer assembly 150 from rotating due to the rotation of the movement unit 120 which is the ball screw.

That is, when the movement unit 120 is not a unit which rotates by a unit such as a motor to horizontally move the housing 140 and the first transducer assembly 150 by the rotation, the horizontal guide 130 may be omitted from the structure 100.

The housing 140 of the present disclosure is coupled with the movement unit 120 and horizontally moves in one direction or the other direction or horizontally reciprocates in one direction and the other direction by the movement unit 120.

Further, the housing 140 may accommodate the first transducer assembly 150 in an inner space for the horizontal movement of the first transducer assembly 150.

The housing 140 may include a spring 141, a first locking groove 142, a second locking groove 143, and an opening 144 to upwardly/downwardly vertically move the first transducer assembly 150 and accommodate the first transducer assembly 150.

The spring 141 may be an elastic member which encloses an outer side of a part of the first transducer assembly 150 which is led in the inner space of the housing 140 through the opening 144.

When the first inclined projection 111 is fitted into the first fitting groove 161 of the fitting unit 160 to be described below, the spring 141 expands to allow the first transducer assembly 150 to upwardly vertically move to an uppermost point.

When the second inclined projection 112 is fitted into the second fitting groove 162 of the fitting unit 160 to be described below, the spring 141 is contracted to allow the first transducer assembly 150 to downwardly vertically move to a lowermost point.

The first locking groove 142 and the second locking groove 143 may be grooves through which a projection (not illustrated) provided at a part of outside of the first transducer assembly 150 which is not illustrated in the drawing, during the upward/downward vertical movement process of the first transducer assembly 150, passes.

The first locking groove 142 is a groove through which the projection of the first transducer assembly 150 passes to maintain the first transducer assembly 150 in an upwardly vertically moved state to the uppermost point when the first transducer assembly 150 upwardly vertically moves to the uppermost point.

The second locking groove 143 is a groove through which the projection of the first transducer assembly 150 passes to maintain the first transducer assembly 150 in a downwardly vertically moved state to the lowermost point when the first transducer assembly 150 downwardly vertically moves to the lowermost point.

The opening 144 may be a groove formed in a lower end of the housing 140 in FIG. 2, to allow the first transducer assembly 150 to be led into the inner space of the housing 140.

The first transducer assembly 150 of the present disclosure has a projection (not illustrated) formed at a part of the outside to maintain an upwardly vertically moved state to the uppermost point or a downwardly vertically moved state to the lowermost point.

At this time, the projection of the first transducer assembly 150 may be a structure which has elasticity to be pressed at an inner side of the housing 140 when the projection does not pass through the first locking groove 142 or the second locking groove 143, to upwardly/downwardly move the first transducer assembly 150.

This is because when the projection of the first transducer assembly 150 does not have elasticity, the projection is not separated from the first locking groove 142 or the second locking groove 143 during the upward/downward vertical movement process of the first transducer assembly 150.

Further, the projection of the first transducer assembly 150 may be compressed to be in contact with an inner wall of the housing 140 when the projection is not parallel to the first locking groove 142 or the second locking groove 143 during the upward/downward vertical movement process of the first transducer assembly 150.

Further, the projection of the first transducer assembly 150 may be compressed to be in contact with an inner wall of the housing 140 during the upward/downward vertical movement process of the first transducer assembly 150 and when the projection is parallel to the first locking groove 142 or the second locking groove 143, passes through the first locking groove 142 or the second locking groove 143 while expanding.

In order to include the projection as described above, in the first transducer assembly 150, it is desirable that a groove (not illustrated) for accommodating the projection to insert or protrude the elastic projection is formed at the part of the outside although not shown in the drawing.

The fitting unit 160 of the present disclosure is disposed above the housing 150 in FIG. 2 so that a lower surface is coupled to an upper end of the first transducer assembly 150.

The fitting unit 160 includes a first fitting groove 161 and a second fitting groove 162 to upwardly and downwardly vertically move the first transducer assembly 150.

The first fitting groove 161 is a groove formed at one side of the fitting unit 160 and is connected to the first inclined projection 111 for upward vertical movement of the first transducer assembly 150.

The second fitting groove 162 is a groove formed at the other side of the fitting unit 160 and is connected to the second inclined projection 112 for downward vertical movement of the first transducer assembly 150.

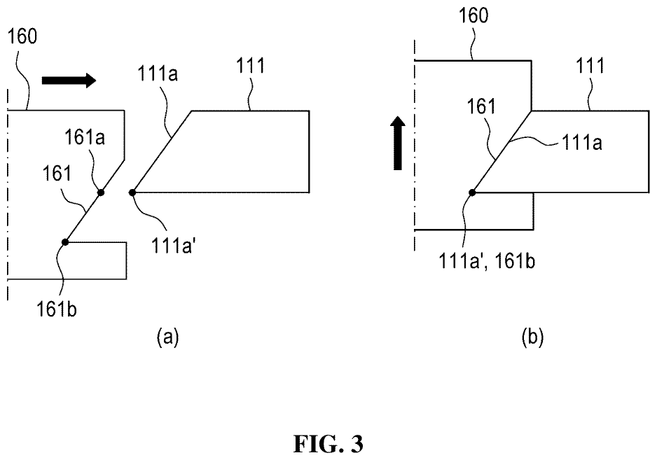

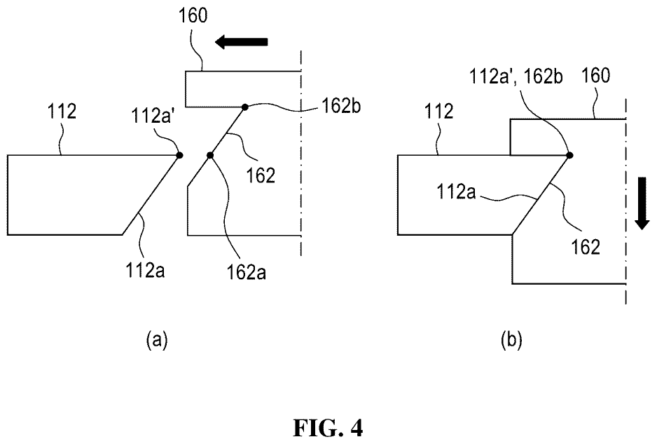

As described above, the connection of the first inclined projection 111 and the first fitting groove 161 and the connection of the second inclined projection 112 and the second fitting groove 162 for upward/downward vertical movement of the first transducer assembly 150 is as illustrated in FIGS. 3 and 4.

FIG. 3 is a view illustrating a fitting process of a first inclined projection and a first fitting groove for upward vertical movement of a first transducer assembly and FIG. 4 is a view illustrating a fitting process of a second inclined projection and a second fitting groove for downward vertical movement of a first transducer assembly.

Referring to FIG. 3A, when the housing 140 and the first transducer assembly 150 horizontally move in one direction by the movement unit 120, an upwardly inclined surface 111a of the first inclined projection 111 is formed on one surface which is opposite to one side of the fitting unit 160 at which the first fitting groove 161 is formed.

The upwardly inclined surface 111a is inclined to be spaced apart from one side of the fitting unit 160 toward the upward direction of FIG. 3A from an end point 111a′ protruding to one side of the fitting unit 160.

During the horizontal movement in one direction of the housing 140 and the first transducer assembly 150 by the movement unit 120, in the fitting unit 160, the first contact point 161a of the first fitting groove 161 and an end point 111a′ of the upwardly inclined surface 111a are in contact with each other.

Referring to FIG. 3B, when the end point 111a′ and the first contact point 161a are in contact with each other, the fitting unit 160 upwardly vertically moves the first transducer assembly 150 coupled to a lower side portion while upwardly sliding by a pressure generated from the first inclined projection 111 fixed to the first frame 110a which applies a force in a horizontal movement energy in one direction of the movement unit 120 which horizontally moves the housing 140 and the first transducer assembly 150 in one direction and an opposite direction to the horizontal movement energy in one direction.

When the fitting unit 160 upwardly slides to upwardly vertically move the first transducer assembly 150, the second contact point 161b of the first fitting groove 161 and an end point 111a′ of the upwardly inclined surface 111a are in contact with each other and at this time, the first inclined projection 111 is fitted into the first fitting groove 161.

As illustrated in FIG. 3B, the first transducer assembly 150 of the present disclosure completes the upward vertical movement to the uppermost point when the first inclined projection 111 is fitted into the first fitting groove 161.

Referring to FIG. 4A, when the housing 140 and the first transducer assembly 150 horizontally move in the other direction by the movement unit 120, a downwardly inclined surface 112a of the first inclined projection 111 is formed on the other surface which is opposite to the other side of the fitting unit 160 at which the second fitting groove 162 is formed.

The downwardly inclined surface 112a is inclined to be spaced apart from the other side of the fitting unit 160 toward the downward direction of FIG. 4A from an end point 112a′ protruding to the other side of the fitting unit 160.

During the horizontal movement in the other direction of the housing 140 and the first transducer assembly 150 by the movement unit 120, in the fitting unit 160, a third contact point 162a of the second fitting groove 162 and an end point 112a′ of the downwardly inclined surface 112a are in contact with each other.

Referring to FIG. 4B, when the end point 112a′ and the third contact point 162a are in contact with each other, the fitting unit 160 downwardly vertically moves the first transducer assembly 150 coupled to a lower side portion while downwardly sliding by a pressure generated from the second inclined projection 112 fixed to the second frame 110b which applies a force in a horizontal movement energy in the other direction of the movement unit 120 which horizontally moves the housing 140 and the first transducer assembly 150 in the other direction and an opposite direction to the horizontal movement energy in the other direction.

When the fitting unit 160 downwardly slides to downwardly vertically move the first transducer assembly 150, a fourth contact point 162b of the second fitting groove 162 and an end point 112a′ of the downwardly inclined surface 112a are in contact with each other and at this time, the second inclined projection 112 is fitted into the second fitting groove 162.

As illustrated in FIG. 4B, the first transducer assembly 150 of the present disclosure completes the upward vertical movement to the lowermost point when the second inclined projection 112 is fitted into the second fitting groove 162.

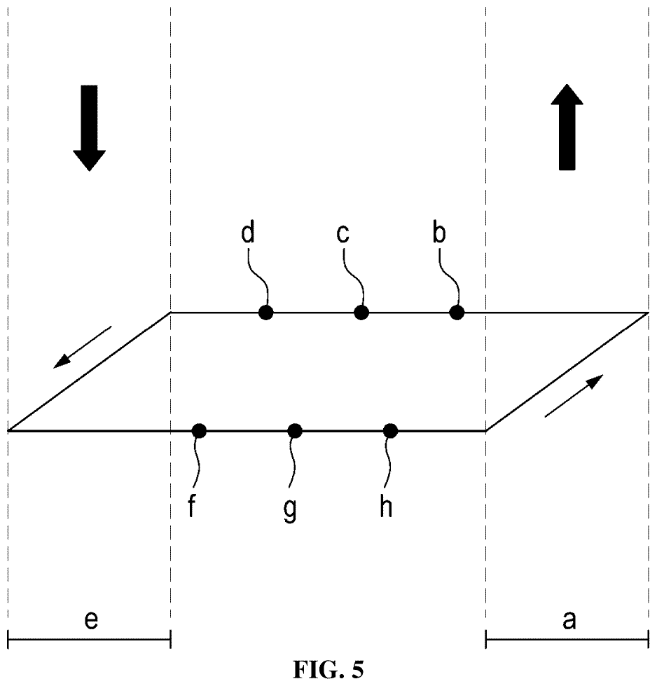

Hereinafter, horizontal movement and upward/downward vertical movement process of a first transducer assembly 150 will be described in detail with reference to FIG. 5.

FIG. 5 is a view for explaining horizontal movement and upward/downward vertical movement of a first transducer assembly.

Referring to FIG. 5, the first transducer assembly 150 of the present disclosure enters a first section (a) for upward vertical movement while horizontally moving in one direction toward the inside of the first frame 110a in a downwardly vertically moved state to the lowermost point and upwardly vertically moves by upwardly sliding by the pressure after the first inclined projection 111 is in contact with the first fitting groove 161 and is located at the uppermost point when the first inclined projection is fitted into the fitting groove 161.

At this time, when the first transducer assembly 150 is disposed at the uppermost point, it means that a lower end of the first transducer assembly 150 is disposed in a coordinate corresponding to the uppermost point in FIG. 2.

Further, when the first transducer assembly 150 upwardly vertically moves to be disposed at the uppermost point, the first transducer assembly is in contact with a lower end of the housing 140 in which the opening 144 is formed.

The surgical instrument for irradiating a focused ultrasound wave on a skin of a subject temporarily stops a process of irradiating a focused ultrasound wave on the skin of the subject until the first transducer assembly reaches the uppermost point from the time when the first transducer assembly starts the upward vertical movement in the first section (a).

When the upward vertical movement to the uppermost point is completed in the first section (a), the first transducer assembly 150 enters a second section (b to d) while maintaining a state disposed in the uppermost point to horizontally move in the other direction toward the inside of the second frame 110b.

At this time, the first transducer assembly 150 maintains the horizontal movement in the other direction to enter a third section (e) for downward vertical movement in the second section (b to d) or horizontally reciprocate in the second section (b to d).

Further, the surgical instrument irradiates the focused ultrasound wave onto the skin of the subject while the first transducer assembly 150 which completes the upward vertical movement horizontally moves in the second section (b to d). When the first transducer assembly 150 horizontally reciprocates in the second section (b to d), a focused ultrasound wave irradiating time when the first transducer assembly 150 is at the uppermost point is increased.

When the first transducer assembly 150 enters the third section (e), the first transducer assembly 150 downwardly slides by the pressure after the second inclined projection 112 is in contact with the second fitting groove 162 to downwardly vertically move and is disposed at the lowermost point when the second inclined projection 112 is fitted into the second fitting groove 162.

At this time, when the first transducer assembly 150 is disposed at the lowermost point, it means that a lower end of the first transducer assembly 150 is disposed in a coordinate corresponding to the lowermost point in FIG. 2.

Further, when the first transducer assembly 150 downwardly vertically moves to be disposed at the lowermost point, the first transducer assembly is disposed as illustrated in FIG. 2.

The surgical instrument may temporarily stop the process of irradiating focused ultrasound wave onto the skin of the subject until it reaches the lowermost point from the time when the first transducer assembly 150 starts the downward vertical movement in the third section (e).

When the downward vertical movement to the lowermost point is completed in the third section (e), the first transducer assembly 150 enters a fourth section (f to h) while maintaining a state disposed in the lowermost point to horizontally move in one direction toward the inside of the first frame 110a.

At this time, the first transducer assembly 150 maintains the horizontal movement in one direction to enter the first section (a) for upward vertical movement in the fourth section (f to h) or horizontally reciprocate in the fourth section (f to h).

That is, the first transducer assembly 150 of the present disclosure horizontally moves and upward/downward vertically moves along the first section (a), the second section (b to d), the third section (e), and the fourth section (f to h) and horizontally reciprocates in the second section (b to d) and the fourth section (f to h) until the skin treatment of the subject ends from starting of the skin treatment.

In the meantime, the surgical instrument irradiates the focused ultrasound wave onto the skin of the subject while the first transducer assembly 150 which completes the downward vertical movement horizontally moves in the fourth section (f to h). When the first transducer assembly 150 horizontally reciprocates in the fourth section (f to h), a focused ultrasound wave irradiating time when the first transducer assembly 150 is at the lowermost point is increased.

The surgical instrument irradiates the focused ultrasound onto the skin of the subject while the first transducer assembly 150 horizontally moves in an upward/downward vertically moved state to irradiate the focused ultrasonic onto the skin of the subject at a plurality of depths.

The surgical instrument of the present disclosure is a device for irradiating an ultrasonic wave on a skin of the subject at a plurality of depths and may be a cartridge and a handpiece to which the structure 100 is applied (or included).

Here, the handpiece is a user interface which allows an operator to irradiate the focused ultrasound wave onto the skin of the subject through manipulation and is held by the operator. The cartridge is a component which is coupled to the handpiece to be recycled or replaced to adjust a focal depth and an intensity of the focused ultrasound wave to be irradiated onto the skin of the subject.

At this time, when the structure 100 is applied to the cartridge, a component of the structure 100 excluding a motor (not illustrated) and a rotary shaft (not illustrated) of the motor may be embedded in the cartridge.

The first transducer assembly 150 serves as a traction shaft to move a transducer (not illustrated) using a driving force of a motor (not illustrated) embedded in the handpiece so that the structure 100 irradiates a focused ultrasound wave generated from the transducer (not illustrated) at a plurality of depths.

In the present disclosure, when the first transducer assembly 150 uses the driving force of the motor (not illustrated), it means that a torque of the rotary shaft (not illustrated) of the motor is used. A method of using the torque of the rotary shaft (not illustrated) of the motor is not limited, and in the exemplary embodiment, a torque of the motor (not illustrated) may be used as it will be described below.

As a specific example, the movement unit 120 rotates with a torque of the motor (not illustrated) through the interaction of a first frictional plate (not illustrated) which is embedded in the handpiece to receive a torque from the rotary shaft (not illustrated) of the motor and a second frictional plate (not illustrated) provided at the end of the movement unit 120 so that the first transducer assembly 150 horizontally moves and upwardly/downwardly vertically moves by following the horizontal movement of the housing 140 and the upward/downward vertical movement of the fitting unit 160 although not shown in the drawing.

In the meantime, the transducer (not illustrated) is embedded in a cartridge to which the structure 100 is applied and is coupled to one side of the first transducer assembly 150 to horizontally move and upwardly/downwardly vertically move by following the horizontal movement and upward/downward vertical movement of the first transducer assembly 150. The transducer generates a focused ultrasound wave while performing a skin surgical method of the subject to irradiate the focused ultrasound wave onto the skin of the subject.

Further, even though the cartridge is not illustrated in the drawing, a transmission opening (not illustrated) which allows a focused ultrasound wave to pass through a contact surface which is in contact with the skin of the subject is formed to irradiate a focused ultrasound wave generated from the transducer (not illustrated) onto the skin of the subject.

In contrast, if the structure 100 is applied to the handpiece, components of the structure 100 excluding a second transducer assembly (not illustrated) to be described below is embedded in the handpiece and the first transducer assembly 150 serves as a first traction shaft to move the transducer (not illustrated) using a driving force of the motor (not illustrated) embedded in the handpiece and is coupled to the second transducer assembly (not illustrated) included in the cartridge.

The second transducer assembly (not illustrated) is coupled to the first transducer assembly 150 to follow the horizontal movement and upward/downward vertical movement of the first transducer assembly 150 and thus serves as a second traction shaft to move the transducer (not illustrated).

In the meantime, the transducer (not illustrated) is embedded in a cartridge tin response to the structure 100 which is applied to the handpiece and is coupled to the second transducer assembly (not illustrated) to horizontally move and upwardly/downwardly vertically move by following the horizontal movement and upward/downward vertical movement of the second transducer assembly (not illustrated). The transducer generates a focused ultrasound wave while performing a skin surgical method of the subject to irradiate the focused ultrasound wave onto the skin of the subject.

Even though the handpiece is not illustrated in the drawing, in order to allow the first transducer assembly 150 to move and be coupled with the second transducer assembly (not illustrated) included in the cartridge, a binding opening (not illustrated) with a diameter through which the first transducer assembly 150 or the second transducer assembly (not illustrated) passes to be movable is formed on a binding surface with the cartridge.

Additionally, when the structure 100 is applied to the handpiece, as it is applied to the cartridge, a transmission opening (not illustrated) through which the focused ultrasound wave passes is formed in the cartridge.

Further, even though it is not illustrated in the drawing, when the movement unit 120 is a ball screw which rotates with a power of the motor (not illustrated), the surgical instrument of the present disclosure includes an encoder which measures a rotation angle of the motor (not illustrated).

Further, the handpiece 11 which is implemented in a wired or wireless manner includes a display (not illustrated) which visually displays a rotation angle of the motor (not illustrated) measured by the encoder.

However, the display is not limited to be included in the handpiece 11, and based on a time when the handpiece 11 is implemented in a wired manner, the display may be a component provided in a separate output device (not illustrated) connected to the handpiece 11.

At this time, the encoder measures the rotation angle of the motor (not illustrated) to accurately calculate a position coordinate (X, Y) of the first transducer assembly 150.

To be more specific, as the horizontal movement and upward/downward vertical movement of the first transducer assembly 150 is accurately synchronized with the rotation angle of the movement unit 120 which is a ball screw due to the structure of the structure 100 and the movement unit 120 rotates by the power of the motor (not illustrated) to be accurately synchronized with the motor (not illustrated), the encoder accurately calculates the position coordinate (X, Y) of the first transducer assembly 150 based on the rotation angle of the motor (not illustrated).

Further, the display may visually display not only the rotation angle of the motor (not illustrated) but also the position coordinate (X, Y) of the first transducer assembly 150.

As described above, the surgical instrument including the encoder accurately calculates the position coordinate (X, Y) of the first transducer assembly 150 so that the surgical instrument irradiates the focused ultrasound wave onto the skin of the subject at a plurality of depths in a desired position according to the operator's manipulation during the horizontal movement and upward/downward vertical movement of the first transducer assembly 150.

### 2) Second Embodiment

Hereinafter, a second embodiment which is a modified example of the first embodiment described above in detail with reference to the accompanying drawings will be described in detail and it is desirably understood that technical features which will not be mentioned in the second embodiment are the same as the first embodiment.

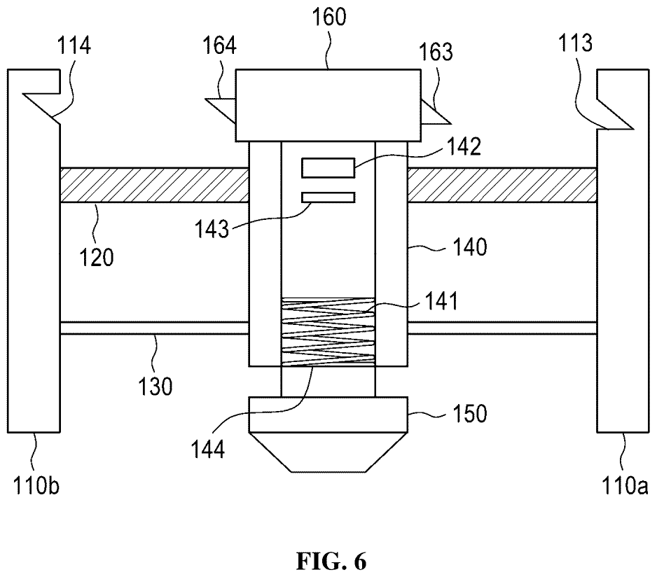

FIG. 6 is a schematic diagram of an inclined projection based structure of a surgical instrument for irradiating a focused ultrasound wave at a plurality of depths according to another exemplary embodiment of the present disclosure.

Referring to FIG. 6, in the structure 100 of the present disclosure, as compared with the first embodiment, a structure of one pair of frames 110 is modified in the second embodiment.

To be more specific, unlike the first embodiment in which the first inclined projection 111 is formed, in the first frame 110a, a first insertion groove 113 is formed in the second embodiment. Further, unlike the first embodiment in which the second inclined projection 112 is formed, in the second frame 110b, a second insertion groove 114 is formed in the second embodiment.

Further, in the structure 100 of the present disclosure, as compared with the first embodiment, a structure of the fitting unit 160 is modified in the second embodiment.

Specifically, unlike the first embodiment in which the first fitting groove 161 and the second fitting groove 162 are formed in the fitting unit 160, a first projection 163 and a second projection 164 are formed.

At this time, when the first transducer assembly 150 horizontally moves in one direction toward the inside of the first frame 110a, the fitting unit 160 upwardly slides by the pressure after the first projection 163 is in contact with the first insertion groove 113 so that the first projection 163 is fitted into the first insertion groove 113.

Further, when the first transducer assembly 150 horizontally moves in the other direction toward the inside of the second frame 110b, the fitting unit 160 downwardly slides by the pressure after the second projection 164 is in contact with the second insertion groove 114 so that the second projection 164 is fitted into the second insertion groove 114.

### Surgical Method Hereinafter, a process of a surgical method S100 using the structure 100 of the present disclosure will be described in detail with reference to the accompanying drawings.

Further, in the following surgical method S100, it is assumed that the structure 100 is implemented in the form of the first embodiment illustrated in FIGS. 1 to 5.

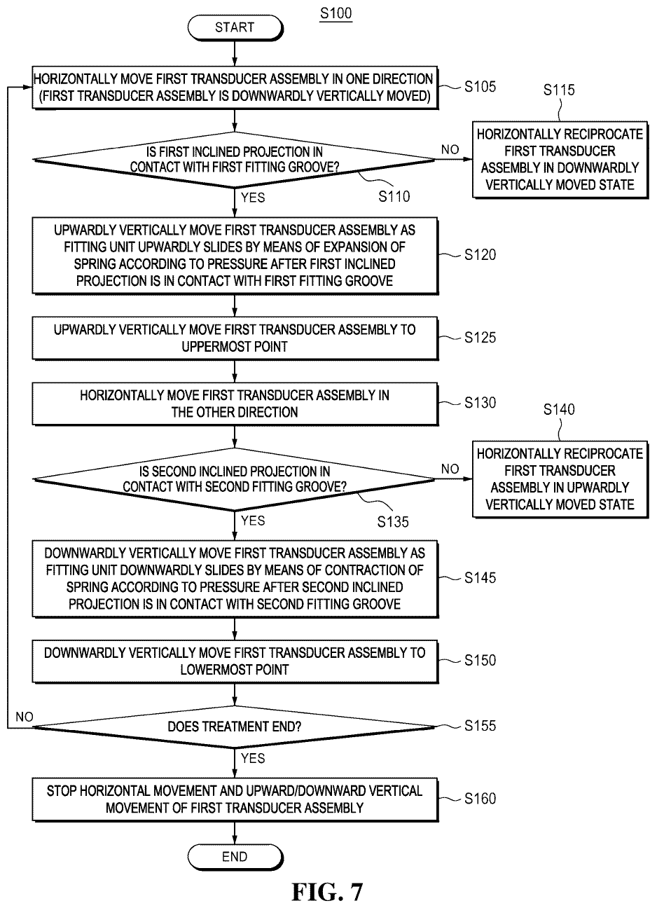

FIG. 7 is a flowchart illustrating processes of a surgical method using an inclined projection based structure of a surgical instrument for irradiating a focused ultrasound wave at a plurality of depths according to an exemplary embodiment of the present disclosure.

Referring to FIG. 7, the surgical method S100 of the present disclosure is a surgical process for improving a skin state of a subject by irradiating a focused ultrasound wave onto a skin of the subject at a plurality of depths and is performed in the order of a first horizontal movement step S105, a first contact step S110, a first horizontal reciprocation step S115, a contact and upward slide step S120, an upward vertical movement step S125, a second horizontal movement step S130, a second contact step S135, a second horizontal reciprocation step S140, a contact and downward side step S145, a downward vertical movement step S150, a step S145->S155 of determining whether a treatment ends, and a horizontal movement and upward/downward vertical movement stop step S150->S160 until the skin treatment of the subject ends from starting of the skin treatment.

Further, the surgical method S100 of the present disclosure will be described by assuming that the first transducer assembly 150 accommodated in the inner space of the housing 140 completes downward vertical movement to the lowermost point.

In the first horizontal movement step S105, the first transducer assembly 150 may horizontally move in one direction by a movement unit 120 which is coupled to the housing 140 to control the horizontal movement of the first transducer assembly 150.

In the first contact step S110, based on a time when the horizontal movement of the first transducer assembly 150 in one direction is completed during the process of horizontally moving the first transducer assembly in one direction by the movement unit 120, the first inclined projection 111 provided in the first frame 110a may be in contact or may not be in contact with the first fitting groove 161 of the fitting unit 160 coupled with the first transducer assembly 150.

At this time, based on a time when the horizontal movement of the first transducer assembly 150 in one direction is completed, if the first inclined projection 111 is not in contact with the first fitting groove 161 (NO in S110), the first transducer assembly 150 may horizontally reciprocate by horizontally moving in the other direction by the movement unit 120 in a downwardly vertically moved state in step S115.

In the first horizontal reciprocation step S115, after completing the horizontal movement in the other direction by the movement unit 120 in a downwardly vertically moved state, the first transducer assembly 150 may horizontally move in one direction again, and also horizontally move in one direction again and then horizontally move in the other direction to horizontally reciprocate.

In contrast, based on a time when the horizontal movement of the first transducer assembly 150 in one direction is completed, if the first inclined projection 111 is in contact with the first fitting groove 161 (YES in S110), the spring 141 expands by the pressure after the first inclined projection 111 is in contact with the first fitting groove 161 so that the fitting unit 160 upwardly slides to upwardly vertically move the first transducer assembly 150 in step S120.

In the upward vertical movement step S125, the first transducer assembly 150 upwardly vertically moves to the uppermost point as the fitting unit 160 upwardly slides to be changed to an upward vertical movement state.

In the meantime, the surgical instrument irradiates the focused ultrasound wave onto the skin of the subject at a plurality of depths and the focused ultrasound wave irradiating step may be paused during the process of upwardly vertically moving the first transducer assembly 150, like the contact and upward slide step S120 and the upward vertical movement step S125.

In the second horizontal movement step S130, the first transducer assembly 150 which completes the upward vertical movement may horizontally move in the other direction by the movement unit 120.

In the second contact step S135, based on a time when the horizontal movement of the first transducer assembly 150 in the other direction is completed during the process of horizontally moving the first transducer assembly in the other direction by the movement unit 120, the second inclined projection 112 provided in the second frame 110b may be in contact with or may not be in contact with the second fitting groove 162 of the fitting unit 160.

At this time, based on a time when the horizontal movement of the first transducer assembly 150 in the other direction is completed, if the second inclined projection 112 is not in contact with the second fitting groove 162 (No in S135), the first transducer assembly 150 horizontally reciprocates by horizontally moving in one direction by the movement unit 120 in an upwardly vertically moved state in step S140.

In the second horizontal reciprocation step S140, after completing the horizontal movement in one direction by the movement unit 120 in an upwardly vertically moved state, the first transducer assembly 150 may horizontally move in the other direction again, and also horizontally move in the other direction again and then horizontally move in one direction to horizontally reciprocate.

In contrast, based on a time when the horizontal movement of the first transducer assembly 150 in the other direction is completed, if the second inclined projection 112 is in contact with the second fitting groove 162 (YES in S135), the spring 141 contracts by the pressure after the second inclined projection 112 is in contact with the second fitting groove 162 so that the fitting unit 160 downwardly slides to downwardly vertically move the first transducer assembly 150 in step S145.

In the downward vertical movement step S145, the first transducer assembly 150 downwardly vertically moves to the lowermost point as the fitting unit 160 downwardly slides to be changed to a downward vertical movement state.

In the meantime, the surgical instrument pauses the focused ultrasound wave irradiating process during the process of downwardly vertically moving the first transducer assembly 150, like the contact and downward slide step S145 and the downward vertical movement step S150.

In the meantime, the operator may determine whether the skin treatment of the subject ends while performing the steps S105 to S150 of the surgical method S100 of the present disclosure in step S155.

At this time, if the operator determines that the skin treatment of the subject does not end (NO in step S155), the surgical method S100 of the present disclosure may repeat the steps S105 to S150.

In contrast, if the operator determines that the skin treatment of the subject ends (YES in step S155), the first transducer assembly 150 stops the horizontal movement and upward/downward vertical movement S160 and the skin treatment of the subject performed by irradiating a focused ultrasound wave may end.

The structure 100 of the present disclosure provides a surgical instrument with a simple configuration including a structure which adjusts an irradiation depth of a focused ultrasound wave by implementing an upward/downward vertical movement of a transducer assembly 150 while omitting a configuration of an actuator for horizontal movement and upward/downward vertical movement and sets an irradiation depth of the focused ultrasound wave without adding a separate sensor.

Further, the structure 100 of the present disclosure accepts the surgical method of the related art and a focused ultrasound wave is irradiated onto a skin of a subject at a plurality of depths based on the upward/downward vertical movement which occurs when inclined projections 111 and 112 and fitting grooves 161 and 162 are in contact with each other and then slide according to a pressure while the transducer assembly 150 horizontally moves to improve a skin state of the subject based on various surgical methods.

As described above, the detailed description of the exemplary embodiments of the disclosed present disclosure is provided such that those skilled in the art implement and carry out the present disclosure. While the disclosure has been described with reference to the preferred embodiments, it will be understood by those skilled in the art that various changes and modifications of the present disclosure may be made without departing from the spirit and scope of the disclosure. For example, those skilled in the art may use configurations disclosed in the above-described exemplary embodiments by combining them with each other. Therefore, the present disclosure is not intended to be limited to the above-described exemplary embodiments but to assign the widest scope consistent with disclosed principles and novel features.

The present disclosure may be implemented in another specific form within the scope without departing from the technical spirit and essential feature of the present disclosure. Therefore, the detailed description should not restrictively be analyzed in all aspects and should be exemplarily considered. The scope of the present disclosure should be determined by rational interpretation of the appended claims and all changes are included in the scope of the present disclosure within the equivalent scope of the present disclosure. The present disclosure is not intended to be limited to the above-described exemplary embodiments but to assign the widest scope consistent with disclosed principles and novel features. Further, claims having no clear quoting relation in the claims are combined to configure the embodiment or may be included as new claims by correction after application.

1. A structure of a surgical instrument for irradiating a focused ultrasound wave at a plurality of depths, the structure comprising:
a movement unit including a ball screw;
a first frame and a second frame disposed opposite to each other, the first frame including a first inclined projection on a surface of the first frame facing the second frame, the second frame including a second inclined projection on a surface of the second frame facing the first frame, the ball screw passing through the first frame and the second frame;
a first transducer assembly configured to move horizontally or move vertically upward or vertically downward by the movement unit to irradiate a focused ultrasound wave at a plurality of depths, the first inclined projection including an upwardly inclined surface to move the first transducer assembly vertically upward, the second inclined projection including a downwardly inclined surface to move the first transducer assembly vertically downward;
a fitting unit coupled with the first transducer assembly and including a first fitting groove and a second fitting groove, the first fitting groove being configured to slide upwardly while being in contact with the first inclined projection to implement an upward vertical movement of the first transducer assembly by a pressure according to an extent to which the first transducer assembly moves horizontally, the second fitting groove being configured to slide downwardly while being in contact with the second inclined projection to implement a downward vertical movement of the first transducer assembly by the pressure; and
a housing accommodating the first transducer assembly in an inner space of the housing and coupled to the movement unit to horizontally move the first transducer assembly in one direction or in another direction opposite to the one direction by the movement unit or to horizontally reciprocate the first transducer assembly in the one direction and the another direction, the housing having a spring therein, the spring surrounding a part of the first transducer assembly accommodated in the inner space of the housing, the spring being configured to expand or contract by upward or downward sliding of the fitting unit to implement the upward vertical movement or the downward vertical movement of the first transducer assembly,
wherein when the first transducer assembly horizontally moves in the one direction by the movement unit in a state in which the first transducer assembly is moved vertically downward to a lowermost point, the first inclined projection is configured to be in contact with the first fitting groove, and the fitting unit is configured to upwardly slide according to the pressure to move the first transducer assembly vertically upward,
wherein when the first inclined projection is positioned in the first fitting groove, the spring is configured to expand to allow the first transducer assembly to move vertically upward to an uppermost point,
wherein when the first transducer assembly horizontally moves in the another direction by the movement unit in a state in which the first transducer assembly is moved vertically upward to the uppermost point, the second inclined projection is configured to be in contact with the second fitting groove, and the fitting unit is configured to slide downward according to the pressure to move the first transducer assembly vertically downward,
wherein when the second inclined projection is positioned the second fitting groove, the spring is configured to contract to allow the first transducer assembly to move vertically downward to the lowermost point,
wherein the first transducer assembly includes an elastic projection positioned on an outer surface of the first transducer assembly to maintain the first transducer assembly at the uppermost point or at the lowermost point,
wherein the housing includes:
a first locking groove configured to pass therethrough the elastic projection of the first transducer assembly to maintain the first transducer assembly at the uppermost point after the first transducer assembly upwardly vertically moves to the uppermost point; and
a second locking groove configured to pass therethrough the elastic projection of the first transducer assembly to maintain the first transducer assembly at the lowermost point after the first transducer assembly downwardly vertically moves to the lowermost point.

2-8. (canceled)

9. The structure according to claim 1,
wherein the movement unit is configured to rotate by a power received from a motor to horizontally move the first transducer assembly, and
wherein the structure further includes:
a horizontal guide coupled to the housing to guide a horizontal movement of the first transducer assembly to allow the first transducer assembly to horizontally move and block rotation about an axis of the ball screw when the movement unit rotates by the power transmitted from the motor.

10. The structure according to claim 1,
wherein the surgical instrument is a cartridge or a handpiece to which the structure is applicable.

11-14. (canceled)

15. A surgical method of irradiating a focused ultrasound wave at a plurality of depths, the surgical method comprising:
horizontally moving a first transducer assembly accommodated in an inner space of a housing in one direction by a movement unit coupled to the housing in a state in which the first transducer assembly is downwardly vertically moved to a lowermost point;
when a first inclined projection of a first frame is not in contact with a first fitting groove of a fitting unit coupled with the first transducer assembly at a time when the horizontally moving the first transducer assembly in the one direction is completed, horizontally reciprocating the first transducer assembly, wherein the movement unit passes through the first frame;
when the first inclined projection is in contact with the first fitting groove at the time when the horizontally moving the first transducer assembly in the one direction is completed, upwardly vertically moving the first transducer assembly by the fitting unit which upwardly slides on the first inclined projection by an expansion of a spring embedded in the housing by a pressure;
upwardly vertically moving the first transducer assembly further to an uppermost point as the fitting unit upwardly slides;
after the upwardly vertically moving the first transducer assembly to the uppermost point is completed, horizontally moving, by the movement unit, the first transducer assembly in another direction opposite to the one direction while the first transducer assembly is maintained at the uppermost point;
when a second inclined projection of a second frame is not in contact with a second fitting groove of the fitting unit at a time when the horizontally moving the first transducer assembly in the another direction is completed, horizontally reciprocating the first transducer assembly, wherein the movement unit passes through the second frame;
when the second inclined projection is in contact with the second fitting groove at the time when the horizontally moving the first transducer assembly in the another direction is completed, downwardly vertically moving the first transducer assembly by the fitting unit which downwardly slides on the second inclined projection by a contraction of the spring by the pressure;
downwardly vertically moving the first transducer assembly further to the lowermost point as the fitting unit downwardly slides,
wherein the movement unit includes a ball screw,
wherein the first frame and the second frame are disposed opposite to each other, the first frame including the first inclined projection on a surface of the first frame facing the second frame, the second frame including the second inclined projection on a surface of the second frame facing the first frame,
wherein the first inclined projection includes an upwardly inclined surface to move the first transducer assembly vertically upward, and the second inclined projection includes a downwardly inclined surface to move the first transducer assembly vertically downward,
wherein when the first transducer assembly horizontally moves in the one direction by the movement unit in a state in which the first transducer assembly is moved vertically downward to the lowermost point, the first inclined projection is configured to be in contact with the first fitting groove, and the fitting unit is configured to upwardly slide according to the pressure to move the first transducer assembly vertically upward,
wherein when the first inclined projection is positioned in the first fitting groove, the spring is configured to expand to allow the first transducer assembly to move vertically upward to the uppermost point,
wherein when the first transducer assembly horizontally moves in the another direction by the movement unit in a state in which the first transducer assembly is moved vertically upward to the uppermost point, the second inclined projection is configured to be in contact with the second fitting groove, and the fitting unit is configured to slide downward according to the pressure to move the first transducer assembly vertically downward,
wherein when the second inclined projection is positioned the second fitting groove, the spring is configured to contract to allow the first transducer assembly to move vertically downward to the lowermost point,
wherein the first transducer assembly includes an elastic projection positioned on an outer surface of the first transducer assembly to maintain the first transducer assembly at the uppermost point or at the lowermost point,
wherein the housing includes:
a first locking groove configured to pass therethrough the elastic projection of the first transducer assembly to maintain the first transducer assembly at the uppermost point after the first transducer assembly upwardly vertically moves to the uppermost point; and
a second locking groove configured to pass therethrough the elastic projection of the first transducer assembly to maintain the first transducer assembly at the lowermost point after the first transducer assembly downwardly vertically moves to the lowermost point.
